SEQUENCE LISTING (3 sequences)

SEQ ID NO: 1 - DNA, 1090 bp, Komagataeibacter europaeus
  source 1..1090: mol_type = other DNA
gcggctgcttaccatgcagtcgcacgaacctttcggggttagtggcggacgggtgagtaacgcgtagggatctgtccatgggtgggggataactttgggaaactgaagctaataccgcatgacacctgagggtcaaaggcgcaagccgcctgtggaggaacctgcgttcgattagctagttggtggggtaaaggcctaccaaggcgatgatcgatagctggtctgagaggatgatcagccacactgggactgagacacggcccagactcctacgggaggcagcagtggggaatattggacaatgggcgcaagcctgatccagcaatgccgcgtgtgtgaagaaggttttcggattgtaaagcactttcagcggggacgatgatgacggtacccgcagaagaagccccggctaacttcgtgccagcagccgcggtaatacgaagggggcaagcgttgctcggaatgactgggcgtaaagggcgcgtaggcggttgacacagtcagatgtgaaattcctgggcttaacctgggggctgcatttgatacgtggcgactagagtgtgagagagggttgtggaattcccagtgtagaggtgaaattcgtagatattgggaagaacaccggtggcgaaggcggcaacctggctcatgactgacgctgaggcgcgaaagcgtggggagcaaacaggattagataccctggtagtccacgctgtaaacgatgtgtgctggatgttgggtgactttgtcattcagtgtcgtagttaacgcgataagcacaccgcctggggagtacggccgcaaggttgaaactcaaaggaattgacgggggcccgcacaagcggtggagcatgtggtttaattcgaagcaacgcgcagaaccttaccagggcttgacatgcggaggccgtgtccagagatgggcatttctcgcaagagacctccagcacaggtgctgcatggctgtcgtcagctcgtgtcgtgagatgttgggttaagtcccgcaacgagcgcaaccttcgcctttagttgcattccgtttgggtgggcctctaaagaactgccggggacaacccgaggaaaggggaat

SEQ ID NO: 2 - DNA, 20 bp, synthetic construct
  misc_feature 1..20: note = sequence of forward primer for identification of 16S rRNA Komagataeibacter europaeus FMES
  source 1..20: mol_type = other DNA
agagtttgatcmtggctcag

SEQ ID NO: 3 - DNA, 22 bp, synthetic construct
  misc_feature 1..22: note = sequence of reverse primer for identification of 16S rRNA Komagataeibacter europaeus FMES
  source 1..22: mol_type = other DNA
tacggctaccttgttacgactt

(19) United States
(12) Patent Application Publication
(10) Pub. No.: US 2024/0392334 A1
(43) Pub. Date: Nov. 28, 2024

(54) BACTERIAL CELLULOSE-PRODUCING KOMAGATAEIBACTER EUROPAEUS STRAIN FMES AND USE THEREOF

(71) Applicants: Innovation Center of Yangtze River Delta, ZJU, Jiaxing (CN); Zhejiang University, Hangzhou (CN)

(72) Inventors: Qihe CHEN, Jiaxing (CN); Siyu LIU, Jiaxing (CN); Ying SHI, Jiaxing (CN); Qianhui QI, Jiaxing (CN)

(73) Assignees: Innovation Center of Yangtze River Delta, ZJU, Jiaxing (CN); Zhejiang University, Hangzhou (CN)

(21) Appl. No.: 18/414,369

(22) Filed: Jan. 16, 2024

(30) Foreign Application Priority Data
May 22, 2023 (CN) 2023105773768

(51) Int. Cl. C12P 19/04; C12N 1/20; C12R 1/01

(52) U.S. Cl. CPC C12P 19/04; C12N 1/205; C12R 2001/01

(57) ABSTRACT

Provided is a bacterial cellulose-producing Komagataeibacter europaeus strain FMES and use thereof. The strain FMES is deposited in the China Center for Type Culture Collection (CCTCC) with a deposit number of CCTCC NO: M20221627. The strain FMES is isolated from traditional vinegar brewing and can efficiently produce bacterial cellulose through static culture. The strain FMES can also be used to produce a novel, green, and non-toxic degradable biotextile. Therefore, the strain FMES is expected to replace leather, textiles, and films derived from animals, plants, and petrochemicals, and shows a potential to serve as a sustainable alternative of the textiles.

### CROSS-REFERENCE TO RELATED APPLICATION

This patent application claims the benefit of and priority to the Chinese Patent Application No. 202310577376.8 filed with the China National Intellectual Property Administration on May 22, 2023, the disclosure of which is incorporated by reference herein in its entirety as part of the present application.

### REFERENCE TO SEQUENCE LISTING

The instant application contains a Sequence Listing which has been submitted electronically in ASCII format and is hereby incorporated by reference in its entirety. Said ASCII copy, created on Apr. 30, 2024, is named 1047.102US_SL.txt and is 5,608 bytes in size.

### TECHNICAL FIELD

The present disclosure belongs to the technical field of microorganisms, and specifically relates to a bacterial cellulose-producing Komagataeibacter europaeus strain FMES and use thereof.

### BACKGROUND

Chemical pollution caused by the production and disposal of clothes is extremely serious to the environment, and there is an urgent need to find sustainable alternative materials for the textile. It is of great significance to develop sustainable biodegradable bacterial cellulose (BC) through microbial cell metabolism of polymer materials to replace various animal-, plant-, and petrochemical-derived textiles.

BC, as a pure extracellular cellulose produced by a type of microorganisms under certain conditions, belongs to a linear polysaccharide with a high degree of polymerization, and is composed of D-glucopyranose as a basic unit that is ligated through β-1,4 glycosidic bonds. As a novel biomaterial, BC has many excellent properties such as high chemical purity and crystallinity, super hydrophilicity as well as water-holding and moisturizing capabilities, and excellent tensile strength, elastic modulus, and hardness. BC is also controllable during biosynthesis. Moreover, BC shows desirable skin-friendliness, biocompatibility, and biodegradability and is an environmental-friendly and decomposable material. In view of this, BC exhibits excellent application prospects in the fields of nutritional food, biomedicine, electronic devices, textiles, cosmetics, papermaking, and coatings.

At present, researchers have found that a variety of bacteria can produce extracellular cellulose, including: Gluconacetobacter, Acetobacter, Rhizobium, Agrobacterium, Achromobacter, Alcaligenes, Bacillus, Pseudomonas, Sarcinia, Salmonella, Enterobacter, and Aerobacter. However, there have been no reports on BC production by Komagataeibacter europaeus.

### SUMMARY

In view of this, an objective of the present disclosure is to provide a Komagataeibacter europaeus strain FMES, which has a desirable function of BC production.

The present disclosure provides a Komagataeibacter europaeus strain FMES with a deposit number of CCTCC NO: M20221627.

The present disclosure further provides a BC-producing microbial inoculant including the Komagataeibacter europaeus strain FMES and an auxiliary material.

The present disclosure further provides the use of the Komagataeibacter europaeus strain FMES or the microbial inoculant in production of BC.

Preferably, BC is fermented by static culture.

The present disclosure further provides the use of the Komagataeibacter europaeus strain FMES or the microbial inoculant in production of a biotextile.

The present disclosure further provides a method for producing BC based on the Komagataeibacter europaeus strain FMES or the microbial inoculant, including the following steps:
inoculating a seed solution of the Komagataeibacter europaeus strain FMES into a fermentation medium to allow for static culture to collect the BC.

Preferably, the static culture is conducted at 27° C. to 33° C.;
the static culture is conducted for 7 d to 15 d; and
the seed solution of the Komagataeibacter europaeus strain FMES is inoculated at an inoculum size of 5% to 10% during the static culture.

Preferably, the fermentation medium includes the following components: 10 g/L to 30 g/L of glucose, 3 g/L to 8 g/L of peptone, 3 g/L to 8 g/L of yeast extract, 1.5 g/L to 4.5 g/L of disodium hydrogen phosphate, and 0.8 g/L to 1.5 g/L of citric acid, and has a pH value of 5.0 to 7.0.

Preferably, a preparation process of the seed solution of the Komagataeibacter europaeus strain FMES includes: inoculating an activated Komagataeibacter europaeus strain FMES into a liquid medium to allow for shaking culture;
the shaking culture is conducted at 27° C. to 33° C. for 36 h to 60 h;
the shaking culture is conducted at 120 rpm to 180 rpm; and
the shaking culture is conducted until the activated Komagataeibacter europaeus strain FMES has an OD₆₀₀ value of 0.8 to 1.0.

Preferably, the collected BC is further subjected to impurity removal;
the impurity removal includes: slowly rinsing BC with deionized water three times, immersing BC in a NaOH solution, and then rinsing BC with deionized water until a resulting effluent reaches a neutral pH value; and

BC is immersed in the NaOH solution with a concentration of 0.5 mol/L to 1.2 mol/L for 18h to 36 h.

The present disclosure provides a Komagataeibacter europaeus strain FMES with a deposit number of CCTCC NO: M20221627. In the present disclosure, a novel BC-producing Komagataeibacter europaeus strain FMES is obtained by isolation and screening. The strain FMES can produce BC through static culture. The strain FMES can also be used to produce a novel, green, and non-toxic degradable biotextile. Therefore, the strain FMES is expected to replace leather, textiles, and films derived from animals, plants, and petrochemicals, and then serves as a sustainable alternative of textiles.

### BRIEF DESCRIPTION OF THE DRAWINGS

FIG. 1 shows the colony appearance of the Komagataeibacter europaeus strain FMES on an HS (Hestrin-Schramm) plate medium in Example 1 of the present disclosure;

FIG. 2 shows a BLAST alignment result of a 16S rDNA sequence of the Komagataeibacter europaeus strain FMES in Example 2 of the present disclosure;

FIG. 3 shows a static fermentation photo of the Komagataeibacter europaeus strain FMES in an Erlenmeyer flask in Example 3 of the present disclosure;

FIG. 4 shows a photo of a fermentation product of the Komagataeibacter europaeus strain FMES in Example 4 of the present disclosure;

FIG. 5 shows an infrared absorption spectrum of BC produced by the Komagataeibacter europaeus strain FMES in Example 4 of the present disclosure;

FIG. 6 shows the appearance of the Komagataeibacter europaeus strain FMES growing around a mold structure in Example 5 of the present disclosure; and

FIG. 7 shows a physical picture of a biotextile produced by the Komagataeibacter europaeus strain FMES in Example 5 of the present disclosure.

### DEPOSIT OF BIOLOGICAL MATERIAL

The Komagataeibacter europaeus strain FMES was deposited in the China Center for Type Culture Collection (CCTCC) in Wuhan University, China on Oct. 21, 2022, with a deposit number of CCTCC NO: M20221627.

### DETAILED DESCRIPTION OF THE EMBODIMENTS

The present disclosure provides a Komagataeibacter europaeus strain FMES with a deposit number of CCTCC NO: M20221627.

In the present disclosure, the Komagataeibacter europaeus strain FMES has milky white, irregular oblate, smooth and convex colonies. Through 16SrDNA molecular identification, the Komagataeibacter europaeus strain FMES has a sequence shown in SEQ ID NO: 1 and is determined to be the Komagataeibacter europaeus strain FMES through alignment with the NCBI database.

In the present disclosure, a culture method of the Komagataeibacter europaeus strain FMES preferably includes the following steps:
inoculating the Komagataeibacter europaeus strain FMES on an HS plate or slant medium to allow activation culture under static conditions; and inoculating a resulting activated Komagataeibacter europaeus strain FMES into a liquid medium to allow for shaking culture to obtain a seed solution of the Komagataeibacter europaeus strain FMES.

In the present disclosure, the HS plate or slant medium preferably includes: 10 g/L to 30 g/L of glucose, 3 g/L to 8 g/L of peptone, 3 g/L to 8 g/L of yeast extract, 1.5 g/L to 4.5 g/L of disodium hydrogen phosphate, 0.8 g/L to 1.5 g/L of citric acid, and 13 g/L to 18 g/L of agar, and has a pH value of 5.0 to 7.0; more preferably includes: 20 g/L of glucose, 5 g/L of peptone, 5 g/L of yeast extract, 2.75 g/L of disodium hydrogen phosphate, 1.155 g/L of citric acid, and 15 g/L of agar, and has a pH value of 6.0. The static culture is conducted preferably at 27° C. to 33° C. for 2 d to 4 d, more preferably at 30° C. for 3 d. The shaking culture is conducted preferably at 27° C. to 33° C. and 120 rpm to 180 rpm for 36 h to 60 h, more preferably at 30° C. and 150 rpm for 48 h.

The present disclosure further provides a BC-producing microbial inoculant, including the Komagataeibacter europaeus strain FMES and an auxiliary material.

In the present disclosure, the auxiliary material is selected from the group consisting of a solid phase auxiliary material and a liquid phase auxiliary material. There is no special limitation on the preparation method of the microbial inoculant, and any preparation method of the microbial inoculant well known in the art can be used.

The present disclosure further provides the use of the Komagataeibacter europaeus strain FMES or the microbial inoculant in production of BC.

In the present disclosure, BC is preferably fermented by static culture. The static culture is conducted at preferably 27° C. to 33° C., more preferably 30° C. The static culture is conducted for preferably 7 d to 15 d, more preferably 10 d.

The present disclosure further provides the use of the Komagataeibacter europaeus strain FMES or the microbial inoculant in production of a biotextile. The BC produced by the Komagataeibacter europaeus strain FMES is tested by Fourier transform infrared spectrometer for BC. The test results show that BC has infrared absorption consistent with the characteristic absorption of cellulose and can be used in the production and preparation of clothing instead of chemical fibers. The present disclosure further provides a method for producing BC based on the Komagataeibacter europaeus strain FMES or the microbial inoculant, including the following steps:
inoculating a seed solution of the Komagataeibacter europaeus strain FMES into a fermentation medium to allow for static culture to collect BC.

In the present disclosure, a preparation process of the seed solution of the Komagataeibacter europaeus strain FMES is the same as the above-mentioned culture method of the Komagataeibacter europaeus strain FMES and will not be repeated here.

In the present disclosure, the static culture is conducted at preferably 27° C. to 33° C., more preferably 30° C. The static culture is conducted for preferably 7 d to 15 d, more preferably 10 d. The seed solution of the Komagataeibacter europaeus strain FMES is inoculated at an inoculum size of preferably 5% to 10%, more preferably 8% during the static culture. The fermentation medium preferably includes: 10 g/L to 30 g/L of glucose, 3 g/L to 8 g/L of peptone, 3 g/L to 8 g/L of yeast extract, 1.5 g/L to 4.5 g/L of disodium hydrogen phosphate, and 0.8 g/L to 1.5 g/L of citric acid, and has a pH value of 5.0 to 7.0; more preferably includes: 20 g/L of glucose, 5 g/L of peptone, 5 g/L of yeast extract, 2.75 g/L of disodium hydrogen phosphate, 1.155 g/L of citric acid, and has a pH value of 6.0.

In the present disclosure, the static culture is preferably conducted in a polycarbonate square tissue culture bottle sterilized at high temperature and high pressure, with a dimension of 120 mm×120 mm×120 mm, and open fermentation was conducted. The tissue culture bottle is preferably sterilized by preferably immersing in 75% alcohol for 24 h and then conducting UV treatment for 15 min.

In the present disclosure, the collected BC is preferably further subjected to impurity removal; the impurity removal includes: slowly rinsing BC with deionized water three times, immersing BC in a NaOH solution, and then rinsing BC with deionized water until a resulting effluent reaches a neutral pH value. BC is immersed in the NaOH solution with a concentration of preferably 0.5 mol/L to 1.2 mol/L for preferably 18 h to 36 h. BC is dried after impurity removal. The drying is conducted at preferably 24° C. to 26° C., more preferably 25° C.

The BC-producing Komagataeibacter europaeus strain FMES and use thereof provided by the present disclosure will be described in detail in connection with the following examples, but they should not be construed as limiting the claimed scope of the present disclosure.

All experimental methods used in the following examples are conventional methods, unless otherwise specified.

The following examples are all routine aseptic operations on microorganisms, unless otherwise specified.

All materials and reagents used in the following examples may be commercially available, unless otherwise specified.

### Example 1

### Screening Method for BC-Producing Bacteria

1. A film was picked from a surface of traditional brewed vinegar with sterile surgical forceps, washed with sterile saline, cut into pieces, and applied to an HS plate medium to allow for static culture at 30° C. for 5 d. A single colony in the HS plate medium was inoculated into a test tube containing an HS liquid medium to allow for static culture at 30° C. for 7 d until a gel-like film grew on the liquid surface of the HS liquid medium.

2. The gel-like film was clamped and washed 2 times with sterile physiological saline by shaking, cut into pieces and diluted in gradient, and applied on the HS plate medium to allow for inverted culture at 30° C. for 5 d. A single colony significantly larger in size was selected to allow for repeated streak culture 3 times by inverted culture at 30° C. for 3 d. A finally cultivated bacterial lawn was scraped and inoculated into an HS liquid medium to allow shaking culture at 30° C. for 48 h under 150 rpm until OD₆₀₀=0.8-1.0.

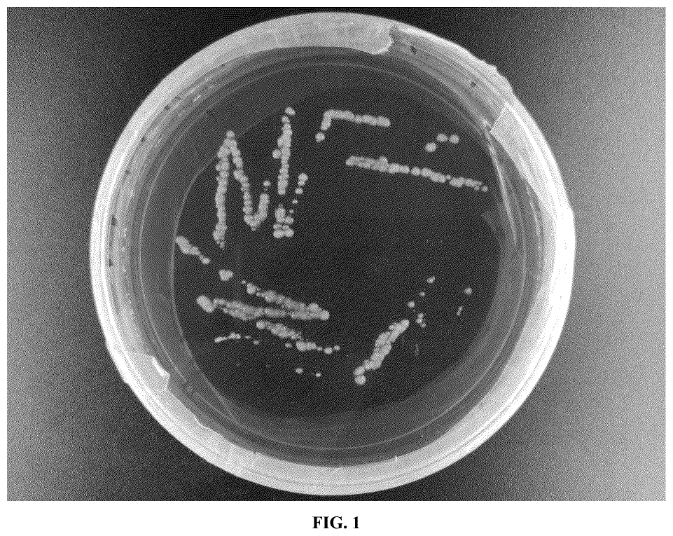

3. The cultured bacterial solution was collected, streaked onto the HS plate medium, and the colony appearance was observed (FIG. 1) to obtain the strain. As shown in FIG. 1, the strain FMES of the present disclosure had milky white, irregular oblate, smooth and convex colonies.

### Example 2

### Molecular Biological Identification of BC-Producing Bacteria

1. Three mL of the cultured bacterial solution was centrifuged in a centrifuge tube at 10,000rpm for 1 min, and a resulting supernatant was aspirated as much as possible to collect bacterial cells.

2. A total bacterial DNA was extracted using a bacterial DNA extraction kit (TIANGEN Biotech (Beijing) Co., Ltd., China). The 16S rDNA gene of the strain FMES was subjected to PCR amplification. Bacterial sequencing primers included: 27F: 5′-AGAGTTTGATCMTGGCTCAG-3′ (SEQ ID NO: 2) and 1492R: 5′-TACGGCTACCTTGTTACGACTT-3′ (SEQ ID NO: 3). PCR amplification conditions were: initial denaturation at 95° C. for 5 min; denaturation at 95° C. for 10 s, annealing at 55° C. for 30 s, extension at 72° C. for 90 s, a total of 35 cycles; final extension at 72° C. for 5 min, and storage at 4° C. A PCR reaction system (50 μL) included: 5 μL of 10× buffer, 5 μL of dNTP, 0.5 μL of 27F, 0.5 μL of 1492R, 0.5 L of Taq enzyme, 0.5 L of template, and 38 μL of double-distilled water.

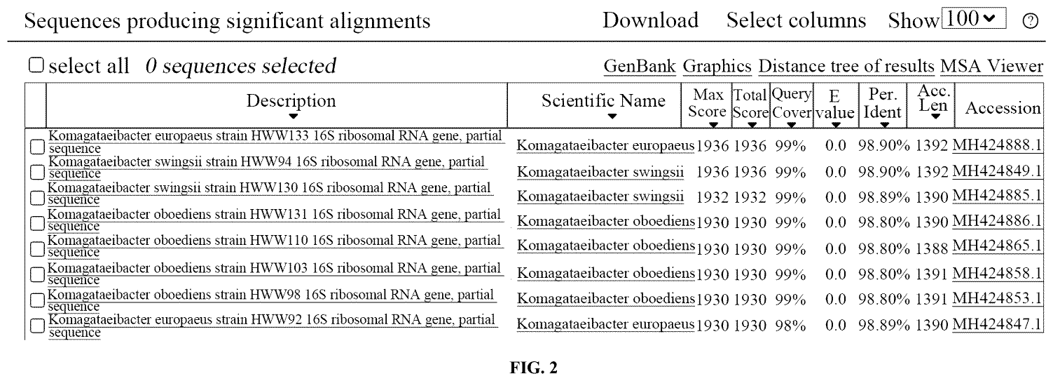

3. The sequencing results are shown in SEQ ID NO: 1 of the sequence listing, and then subjected to BLAST sequence alignment on NCBI. The results are shown in FIG. 2. The results showed hat the 16S rDNA sequence of this bacterium (GCGGCTGCTTACCATGCAGTCGCACGAACCTTTCGGGGTTAGTGGCGGACGGGTGAGT AACGCGTAGGGATCTGTCCATGGGTGGGGGATAACTTTGGGAAACTGAAGCTAATACCG CATGACACCTGAGGGTCAAAGGCGCAAGCCGCCTGTGGAGGAACCTGCGTTCGATTAG CTAGTTGGTGGGGTAAAGGCCTACCAAGGCGATGATCGATAGCTGGTCTGAGAGGATGA TCAGCCACACTGGGACTGAGACACGGCCCAGACTCCTACGGGAGGCAGCAGTGGGGA ATATTGGACAATGGGCGCAAGCCTGATCCAGCAATGCCGCGTGTGTGAAGAAGGTTTTC GGATTGTAAAGCACTTTCAGCGGGGACGATGATGACGGTACCCGCAGAAGAAGCCCCG GCTAACTTCGTGCCAGCAGCCGCGGTAATACGAAGGGGGCAAGCGTTGCTCGGAATGA CTGGGCGTAAAGGGCGCGTAGGCGGTTGACACAGTCAGATGTGAAATTCCTGGGCTTAA CCTGGGGGCTGCATTTGATACGTGGCGACTAGAGTGTGAGAGAGGGTTGTGGAATTCCC AGTGTAGAGGTGAAATTCGTAGATATTGGGAAGAACACCGGTGGCGAAGGCGGCAACC TGGCTCATGACTGACGCTGAGGCGCGAAAGCGTGGGGAGCAAACAGGATTAGATACCC TGGTAGTCCACGCTGTAAACGATGTGTGCTGGATGTTGGGTGACTTTGTCATTCAGTGTC GTAGTTAACGCGATAAGCACACCGCCTGGGGAGTACGGCCGCAAGGTTGAAACTCAAA GGAATTGACGGGGGCCCGCACAAGCGGTGGAGCATGTGGTTTAATTCGAAGCAACGCG CAGAACCTTACCAGGGCTTGACATGCGGAGGCCGTGTCCAGAGATGGGCATTTCTCGCA AGAGACCTCCAGCACAGGTGCTGCATGGCTGTCGTCAGCTCGTGTCGTGAGATGTTGGG TTAAGTCCCGCAACGAGCGCAACCTTCGCCTTTAGTTGCATTCCGTTTGGGTGGGCCTCT AAAGAACTGCCGGGGACAACCCGAGGAAAGGGGAAT, SEQ ID NO: 1) had not less than 98.80% similarity with that of multiple species of the genus Komagataeibacter, and showed a similarity of 98.89% with the 16S rDNA sequence of Komagataeibacter europaeus HWW133 (MH424888.1). In view of this, it was determined that this strain FMES belonged to genus Komagataeibacter and was named Komagataeibacter europaeus strain FMES.

### Example 3

Production of BC by Komagataeibacter europaeus Strain FMES

1. The Komagataeibacter europaeus strain FMES was inoculated on an HS activation plate medium, and the plate was inverted to allow for static culture in a 30° C. incubator for 3 d. Two loops of activated bacteria were picked from the plate with an inoculation loop, and inoculated into 10 mL of HS liquid medium to allow for shaking culture at 30° C. for 48 h under 150 rpm.

2. A mature seed solution with OD₆₀₀=0.8-1.0 was inoculated into a 2,500 mL Erlenmeyer flask containing 750 mL of fermentation medium at an inoculum size of 8% (volume percentage) to allow for static culture in a 30° C. incubator for 10 d.

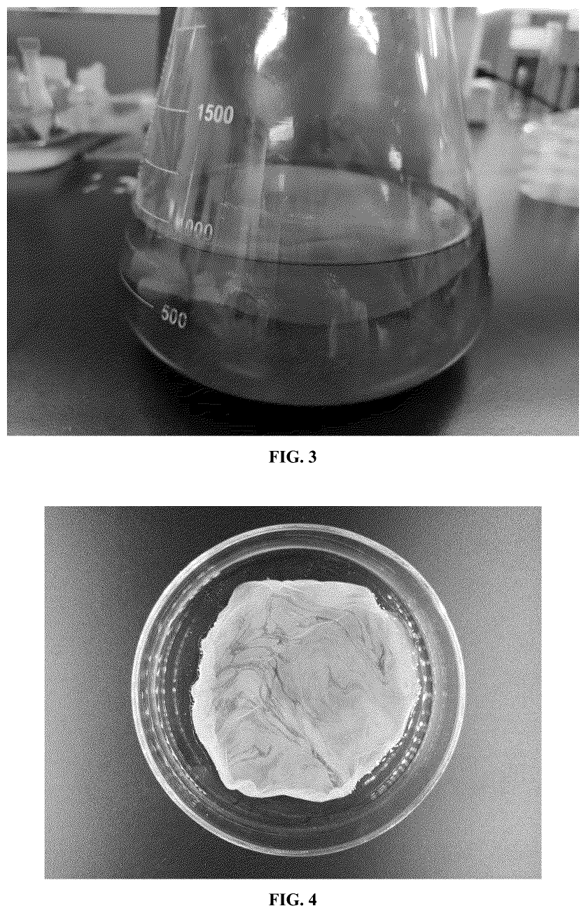

3. A gel-like film produced on a surface of the medium was collected, as shown in FIG. 3, namely the BC.

### Example 4

### Identification of BC

1. The BC in Example 3 was rinsed thoroughly with deionized water 3 times, immersed in a 1 mol/L NaOH solution for 24 h to remove cells and other impurities embedded in the film, the film was rinsed thoroughly with deionized water until the resulting effluent reached a neutral pH value, and the resulting milky white translucent BC film (FIG. 4) was dried at 60° C.

2. The infrared absorption of the dried BC film was measured with a Fourier transform infrared spectrometer at room temperature with a scanning range of (400-4,000) cm⁻¹ and a resolution of 4 cm⁻¹.

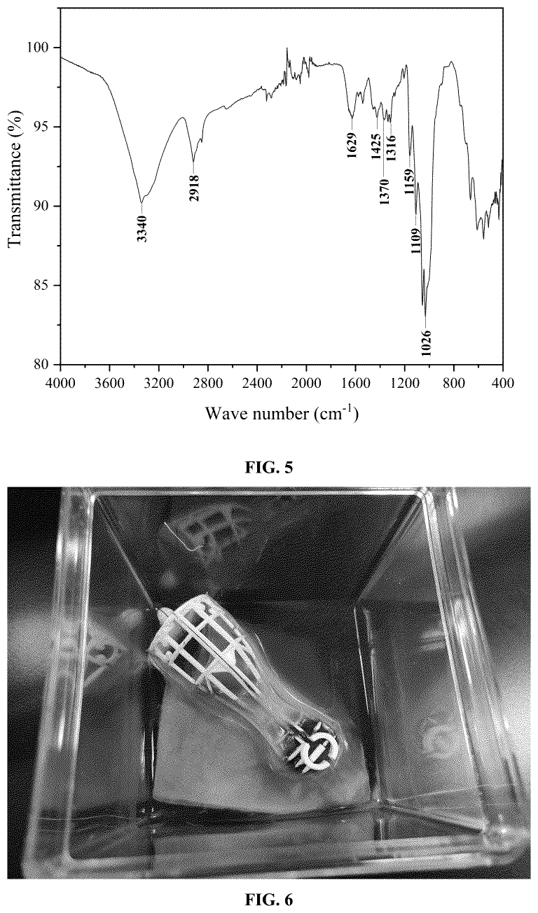

3. The infrared absorption spectrum is shown in FIG. 5. The absorption peak appearing at the wavelength of 3,340 cm⁻¹ was caused by the stretching vibration of O—H; the absorption peak appearing at the wavelength of 2,918 cm⁻¹ was caused by the stretching vibration of the C—H bond; and the absorption peak appearing at the wavelength of 1,629 cm⁻¹ was caused by the H—O—H bending vibration of water molecules adsorbed in cellulose. Multiple absorption peaks appearing at wavelengths (1,200-1,500) cm⁻¹ were caused by C—H in-plane bending and rocking vibration. The multiple absorption peaks appearing at wavelengths (1,000-1,200) cm⁻¹ were mainly caused by the glycosidic bonds between cellulose, the stretching vibration of the C═O bond of the acetal in the molecule, and the stretching vibration of the pyranose ring skeleton. These results indicated that the infrared absorption of the films produced by the Komagataeibacter europaeus strain FMES was consistent with the characteristic absorption of cellulose.

### Example 5

Use of Komagataeibacter europaeus Strain FMES in Production of Biotextile

1. The Komagataeibacter europaeus strain FMES was inoculated on an HS activation plate medium, and the plate was inverted to allow for static culture in a 30° C. incubator for 3 d. Two loops of activated bacteria were picked from the plate with an inoculation loop, and inoculated into 10 mL of HS liquid medium to allow for shaking culture at 30° C. for 48 h at 150 rpm.

2. A mature seed solution with OD₆₀₀=0.8-1.0 was inoculated into a 1,000 mL tissue culture bottle containing 200 mL of fermentation medium as well as a pre-sterilized mold at an inoculum size of 10% (volume percentage) to allow for static culture in a 30° C. incubator for 14 d. The mold had been immersed in 75% alcohol for 24 h and then placed in ultraviolet to allow for sterilization for 15min. As shown in FIG. 6, the Komagataeibacter europaeus strain FMES grew around a structure of the mold.

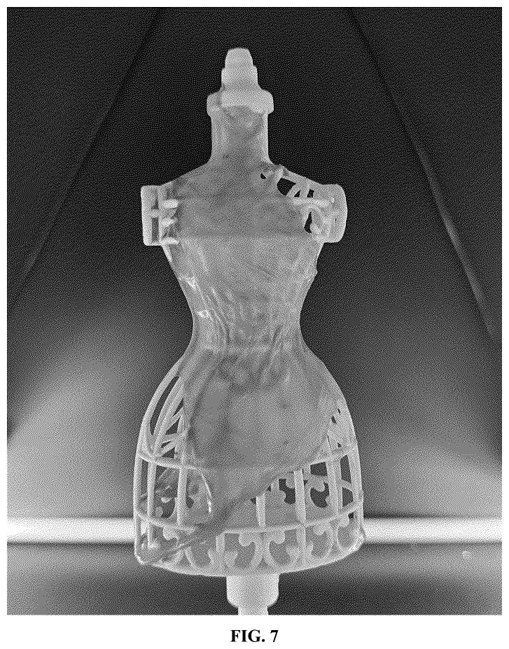

3. The BC textile film was taken out together with the mold, and rinsed slowly 3 times with deionized water, immersed in a 1 mol/L NaOH solution for 24 h to remove cells and other impurities embedded in the film, the film was slowly rinsed with deionized water until the resulting effluent reached a neutral pH value. The film was dried at room temperature of 25±1° C. to obtain the BC shown in FIG. 7, with a dry weight yield of 2.29 g/L.

Described above are merely preferred implementations of the present disclosure. It should be noted that a person of ordinary skill in the art may further make several improvements and modifications without departing from the principle of the present disclosure, but such improvements and modifications should be deemed as falling within the protection scope of the present disclosure.

1-10. (canceled)

11. A bacterial cellulose-producing microbial inoculant, comprising a Komagataeibacter europaeus strain FMES with a deposit number of CCTCC NO: M20221627.

12. The bacterial cellulose-producing microbial inoculant, further comprising an auxiliary material.

13. A method for producing bacterial cellulose, comprising applying the Komagataeibacter europaeus strain FMES according to claim 11.

14. The method according to claim 13, wherein the bacterial cellulose-producing microbial inoculant further comprises an auxiliary material.

15. The method according to claim 13, wherein bacterial cellulose BC is fermented by static culture.

16. A method for producing a biotextile, comprising applying the cellulose-producing microbial inoculant according to claim 12.

17. The method according to claim 16, wherein the bacterial cellulose-producing microbial inoculant further comprises an auxiliary material.

18. A method for producing bacterial cellulose based on the Komagataeibacter europaeus strain FMES according to claim 11, comprising the following steps:
inoculating a seed solution of the Komagataeibacter europaeus strain FMES into a fermentation medium to allow for static culture to collect bacterial cellulose.

19. The method according to claim 18, wherein the bacterial cellulose-producing microbial inoculant further comprises an auxiliary material.

20. The method according to claim 18, wherein the static culture is conducted at 27° C. to 33° C.;
the static culture is conducted for 7 d to 15 d; and
the seed solution of the Komagataeibacter europaeus strain FMES is inoculated at an inoculum size of 5% to 10% during the static culture.

21. The method according to claim 18, wherein the fermentation medium comprises the following components: 10 g/L to 30 g/L of glucose, 3 g/L to 8 g/L of peptone, 3 g/L to 8 g/L of yeast extract, 1.5 g/L to 4.5 g/L of disodium hydrogen phosphate, and 0.8 g/L to 1.5 g/L of citric acid, and has a pH value of 5.0 to 7.0.

22. The method according to claim 18, wherein a preparation process of the seed solution of the Komagataeibacter europaeus strain FMES comprises: inoculating an activated Komagataeibacter europaeus strain FMES into a liquid medium to allow for shaking culture;
the shaking culture is conducted at 27° C. to 33° C. for 36 h to 60 h;
the shaking culture is conducted at 120 rpm to 180 rpm; and
the shaking culture is conducted until the activated Komagataeibacter europaeus strain FMES has an OD₆₀₀ value of 0.8 to 1.0.

23. The method according to claim 18, wherein the collected BC is further subjected to impurity removal;
the impurity removal comprises: slowly rinsing bacterial cellulose with deionized water three times, immersing bacterial cellulose in a NaOH solution, and then rinsing bacterial cellulose with deionized water until a resulting effluent reaches a neutral pH value; and
bacterial cellulose is immersed in the NaOH solution with a concentration of 0.5 mol/L to 1.2 mol/L for 18 h to 36 h.

24. The method according to claim 20, wherein collected bacterial cellulose is further subjected to impurity removal;
the impurity removal comprises: slowly rinsing bacterial cellulose with deionized water three times, immersing bacterial cellulose in a NaOH solution, and then rinsing the BC with deionized water until a resulting effluent reaches a neutral pH value; and
bacterial cellulose is immersed in the NaOH solution with a concentration of 0.5 mol/L to 1.2 mol/L for 18 h to 36 h.

25. The method according to claim 21, wherein collected bacterial cellulose is further subjected to impurity removal;
the impurity removal comprises: slowly rinsing bacterial cellulose with deionized water three times, immersing bacterial cellulose in a NaOH solution, and then rinsing the BC with deionized water until a resulting effluent reaches a neutral pH value; and
the BC is immersed in the NaOH solution with a concentration of 0.5 mol/L to 1.2 mol/L for 18 h to 36 h.

26. The method according to claim 22, wherein collected bacterial cellulose is further subjected to impurity removal;
the impurity removal comprises: slowly rinsing bacterial cellulose with deionized water three times, immersing bacterial cellulose in a NaOH solution, and then rinsing bacterial cellulose with deionized water until a resulting effluent reaches a neutral pH value; and
bacterial cellulose is immersed in the NaOH solution with a concentration of 0.5 mol/L to 1.2 mol/L for 18 h to 36 h.
